(19) United States
(12) Patent Application Publication
(10) Pub. No.: US 2024/0293378 A1
(43) Pub. Date: Sep. 5, 2024

(54) METHODS AND COMPOSITIONS FOR TREATING GORLIN SYNDROME

(71) Applicant: PALVELLA THERAPEUTICS, INC., Wayne, PA (US)

(72) Inventors: Braham SHROOT, Antibes (FR); Kathleen GOIN, Wayne, PA (US); Wesley Harton KAUPINEN, Wayne, PA (US)

(73) Assignee: PALVELLA THERAPEUTICS, INC., Wayne, PA (US)

(21) Appl. No.: 18/572,488

(22) PCT Filed: Jun. 21, 2022
(86) PCT No.: PCT/US22/34347

§ 371 Date: Dec. 20, 2023

Related U.S. Application Data
(60) Provisional application No. 63/212,936, filed on Jun. 21, 2021.
(60) Provisional application No. 63/364,902, filed on May 18, 2022.

(51) Int. Cl. A61K 31/436; A61K 9/00; A61K 9/06; A61K 47/10; A61K 47/12; A61K 47/14; A61K 47/22; A61K 47/38; A61P 17/00; A61P 35/00

(52) U.S. Cl. CPC A61K 31/436; A61K 9/0014; A61K 9/06; A61K 47/10; A61K 47/12; A61K 47/14; A61K 47/22; A61K 47/38; A61P 17/00; A61P 35/00

(57) ABSTRACT

Disclosed herein are compositions and methods to treat Gorlin Syndrome in a subject and preventing basal cell carcinomas in a Gorlin Syndrome subject. In some embodiments, the method comprises administering to the subject an anhydrous rapamycin gel composition disclosed herein. In some embodiments, administration comprises applying the composition as a thin layer on the subject's skin. In some embodiments, the composition is administered once daily.

### CLAIM OF PRIORITY

This application claims the benefit of priority to U.S. Provisional Application No. 63/364,902, filed May 18, 2022, and U.S. Provisional Application No. 63/212,936, filed Jun. 21, 2021, which are incorporated herein by reference in their entirety.

### SUMMARY

The invention is based on the surprising finding from clinical studies that rapamycin can be used for the treatment of Gorlin Syndrome as well as the treatment or prevention of basal cell carcinoma a symptom in subjects with Gorlin Syndrome (also known as basal cell nevus syndrome (BCNS)).

Consequently, there are three aspects of the present invention. The first aspect described herein are methods of treating Gorlin Syndrome in a subject in need thereof comprising topically administering to the subject an anhydrous rapamycin gel composition disclosed herein.

Additional aspects described herein are methods of decreasing the risk of progression or decreasing the risk of developing basal cell carcinoma in a subject with Gorlin Syndrome comprising topically administering to the subject an anhydrous rapamycin gel composition disclosed herein.

The anhydrous rapamycin gel composition disclosed herein topically administered in any of the aspects of the invention may be any that are disclosed herein.

The step of administration in any of the aspects of the present invention may comprise topically applying the anhydrous rapamycin gel composition disclosed herein as a thin layer over the skin of the subject's skin.

The step of administration in any of the aspects of the present invention may comprise topically applying the anhydrous rapamycin gel composition disclosed herein as a thin layer over the skin of the subject's face.

The topical administration of the anhydrous rapamycin gel composition disclosed herein may be once daily, twice daily, or three times daily. Alternatively, the anhydrous rapamycin gel composition disclosed herein may be topically administered 3 times per week, 2 times per week, or 1 time per week.

The subject of any of the aspects of the present invention may have a genetic mutation in patched-1 (PTCH1) gene and may have an anhydrous rapamycin gel composition of rapamycin disclosed herein topically administered to them in accordance with the present invention.

The subject of any of the aspects of the present invention may develop no more than 2 new basal cell carcinomas (BCCs) on the skin in about a 6 month period after topically administering the anhydrous rapamycin gel composition disclosed herein in accordance with the present invention.

The subject of any of the aspects of the present invention may develop no more than 2 new basal cell carcinomas (BCCs) on the skin in about a 12 month period after topically administering the anhydrous rapamycin gel composition disclosed herein in accordance with the present invention.

The subject of any of the aspects of the present invention may develop no more than 2 new basal cell carcinomas (BCCs) on the face in about a 6 month period after topically administering the anhydrous rapamycin gel composition disclosed herein in accordance with the present invention.

The subject of any of the aspects of the present invention may develop no more than 1 new basal cell carcinoma (BCC) on the skin in about a 6 month period after topically administering the anhydrous rapamycin gel composition disclosed herein in accordance with the present invention.

The subject of any of the aspects of the present invention may develop no more than 1 new basal cell carcinoma (BCC) on the face in about a 12 month period after topically administering the anhydrous rapamycin gel composition disclosed herein in accordance with the present invention.

The subject of any of the aspects of the present invention may develop no new basal cell carcinoma (BCC) on the skin in about a 6 month period after topically administering the anhydrous rapamycin gel composition disclosed herein in accordance with the present invention.

The subject of any of the aspects of the present invention may develop no new basal cell carcinoma (BCC) on the face in about a 12 month period after topically administering the anhydrous rapamycin gel composition disclosed herein in accordance with the present invention.

The lesion size (measured as the sum of the longest diameter) of a basal cell carcinoma (BCC) on the skin of the subject of any of the aspects of the present invention may have decreased in size after topically administering the anhydrous rapamycin gel composition disclosed herein in accordance with the present invention.

The subject of any of the aspects of the present invention may see an increase in the time until a new basal cell carcinoma (BCC) biopsy is confirmed after topically administering the anhydrous rapamycin gel composition disclosed herein in accordance with the present invention.

The subject of any of the aspects of the present invention may see improvement in the score on the advanced basal cell carcinoma index (aBCCdex) after topically administering the anhydrous rapamycin gel composition disclosed herein in accordance with the present invention.

The subject of any of the aspects of the present invention may see improvement in the score on the dermatology life quality index (DLQI) after topically administering the anhydrous rapamycin gel composition disclosed herein in accordance with the present invention.

### DETAILED DESCRIPTION

Various aspects now will be described more fully hereinafter. Such aspects may, however, be embodied in many different forms and should not be construed as limited to the embodiments set forth herein: rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

All percentages, parts and ratios are based upon the total weight of the anhydrous rapamycin gel compositions disclosed herein and all measurements made are at about 25° C., unless otherwise specified.

The singular forms “a,” “an,” and “the” include plural referents unless the context clearly dictates otherwise. Thus, for example, reference to a “subject” includes a single subject as well as two or more subjects: reference to an “excipient” includes a single excipient as well as two or more of the same or different excipients, and the like.

As used herein, the term “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation.

The terms “administer,” “administering” or “administration” as used herein refer to either directly administering rapamycin, or pharmaceutically acceptable salt thereof, or an anhydrous rapamycin gel composition described herein to a subject.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. In embodiments or claims where the term comprising is used as the transition phrase, such embodiments can also be envisioned with replacement of the term “comprising” with the terms “consisting of” or “consisting essentially of.”

The term “patient” and “subject” are interchangeable and is a human. In some embodiments, the human is an adult, child or infant. In some embodiments, the patient or subject is an adult human.

The terms “rapamycin” and “sirolimus” are interchangeable and refer to the same drug.

As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms: diminishment of the extent or vigor or rate of development of the condition, disorder or disease: stabilization (i.e., not worsening) of the state of the condition, disorder or disease: delay in onset or slowing of the progression of the condition, disorder or disease: amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

The “weight percent” disclosed is weight-to-weight percent.

### Methods of Use

Disclosed herein are anhydrous rapamycin gel compositions and methods to treat Gorlin Syndrome in a subject and methods to prevent BCC in a Gorlin Syndrome subject. GS patients can present with hundreds of BCCs over their lifetime; consequently, GS patients often experience a significantly diminished quality of life. In addition to proliferative BCC development, other features of GS include macrocephaly, skeletal abnormalities involving the spine, ribs and/or skull, odontogenic keratocysts, medulloblastoma, and/or small depressions in the palms of the hands and soles of the feet.

Disclosed herein are methods of treating Gorlin Syndrome in a subject in need thereof comprising topically administering to the subject an anhydrous rapamycin gel composition disclosed herein.

Additionally disclosed herein are methods of decreasing the risk of progression or decreasing the risk of developing basal cell carcinoma in a subject with Gorlin Syndrome comprising topically administering to the subject an anhydrous rapamycin gel composition disclosed herein.

The anhydrous rapamycin gel composition disclosed herein topically administered in any of the aspects of the invention may be any that are disclosed herein.

The step of administration in any of the aspects of the present invention may comprise topically applying the anhydrous rapamycin gel composition disclosed herein as a thin layer over the skin of the chest, the neck, the scalp, the back, the face, or combinations thereof. Preferably, the step of administration in any of the aspects of the present invention may comprise topically applying the anhydrous rapamycin gel composition disclosed herein as a thin layer over the skin of the subject's face.

The topical administration of the anhydrous rapamycin gel composition disclosed herein according to any of the aspects of the present invention may result in rapamycin reaching the epidermal layer of the skin through absorption. The topical administration of the anhydrous rapamycin gel composition disclosed herein according to any of the aspects of the present invention may result in rapamycin reaching the dermal layer of the skin through absorption. The topical administration of the anhydrous rapamycin gel composition disclosed herein according to any of the aspects of the present invention may result in no significant systemic absorption of rapamycin.

The subject may be at least 18 years of age.

The topical administration of the anhydrous rapamycin gel composition disclosed herein according to any of the aspects of the present invention may be once daily, twice daily, or three times daily. Alternatively, the anhydrous rapamycin gel composition disclosed herein may be topically administered 3 times per week, 2 times per week, 1 time per week, or once every other day. Preferably, the anhydrous rapamycin gel composition disclosed herein may be topically administered once a day.

The topical administration of the anhydrous rapamycin gel composition disclosed herein according to any of the aspects of the present invention may be administered for a period of at least 1 week. The topical administration of the anhydrous rapamycin gel composition disclosed herein according to any of the aspects of the present invention may be administered for a period of at least 2 weeks. The topical administration of the anhydrous rapamycin gel composition disclosed herein according to any of the aspects of the present invention may be administered for a period of at least 3 weeks.

The topical administration of the anhydrous rapamycin gel composition disclosed herein according to any of the aspects of the present invention may be administered for a period of at least 4 weeks. The topical administration of the anhydrous rapamycin gel composition disclosed herein according to any of the aspects of the present invention may be administered for a period of at least 5 weeks. The topical administration of the anhydrous rapamycin gel composition disclosed herein according to any of the aspects of the present invention may be administered for a period of at least 6 weeks. The topical administration of the anhydrous rapamycin gel composition disclosed herein according to any of the aspects of the present invention may be administered for a period of at least 7 weeks. The topical administration of the anhydrous rapamycin gel composition disclosed herein according to any of the aspects of the present invention may be administered for a period of at least 8 weeks. The topical administration of the anhydrous rapamycin gel composition disclosed herein according to any of the aspects of the present invention may be administered for a period of at least 9 weeks. The topical administration of the anhydrous rapamycin gel composition disclosed herein according to any of the aspects of the present invention may be administered for a period of at least 10 weeks. The topical administration of the anhydrous rapamycin gel composition disclosed herein according to any of the aspects of the present invention may be administered for a period of at least 11 weeks. Preferably, the topical administration of the anhydrous rapamycin gel composition disclosed herein according to any of the aspects of the present invention may be administered for a period of at least 12 weeks. More preferably, the topical administration of the anhydrous rapamycin gel composition disclosed herein according to any of the aspects of the present invention may be administered for a period of at least 24 weeks. More preferably, the topical administration of the anhydrous rapamycin gel composition disclosed herein according to any of the aspects of the present invention may be administered for a period of at least 48 weeks. The topical administration of the anhydrous rapamycin gel composition disclosed herein according to any of the aspects of the present invention may be administered for a period of at least 3 months. The topical administration of the anhydrous rapamycin gel composition disclosed herein according to any of the aspects of the present invention may be administered for a period of at least 4 months. The topical administration of the anhydrous rapamycin gel composition disclosed herein composition according to any of the aspects of the present invention may be administered for a period of at least 5 months. The topical administration of the anhydrous rapamycin gel composition disclosed herein according to any of the aspects of the present invention may be administered for a period of at least 6 months. The topical administration of the anhydrous rapamycin gel composition disclosed herein according to any of the aspects of the present invention may be administered for a period of at least 8 months. The topical administration of the anhydrous rapamycin gel composition disclosed herein according to any of the aspects of the present invention may be administered for a period of at least 12 months.

The topical administration of the anhydrous rapamycin gel composition disclosed herein according to any of the aspects of the present invention may be in dosing cycles, wherein the anhydrous rapamycin gel composition disclosed herein is administered for a period of time as described herein and then administration ceases for a period of time and then the administration resumes again for another period of time as described herein. The time of cessation of the administration of the anhydrous rapamycin gel composition disclosed herein may be from about 1 week to about 12 weeks.

The topical administration of the anhydrous rapamycin gel composition disclosed herein according to any of the aspects of the present invention may be through a route selected from topical, transdermal, or percutaneous.

The subject of any of the aspects of the present invention may have a genetic mutation in patched-1 (PTCH1) gene and may have an anhydrous rapamycin gel composition disclosed herein topically administered to them in accordance with the present invention.

The subject of any of the aspects of the present invention may not be taking Hedgehog (Hh) inhibitors and may have an anhydrous rapamycin gel composition disclosed herein topically administered to them in accordance with the present invention.

The subject of any of the aspects of the present invention may not be taking SMO inhibitors and may have an anhydrous rapamycin gel composition disclosed herein topically administered to them in accordance with the present invention.

The methods of treating Gorlin Syndrome in a subject in need thereof comprise topically administering to the affected areas of the subject once daily an anhydrous rapamycin gel composition disclosed herein, wherein treatment is defined as the subject not developing a new BCC in a 6 month period once treatment has started.

The methods of treating Gorlin Syndrome in a subject in need thereof comprise topically administering to the affected areas of the subject once daily an anhydrous rapamycin gel composition disclosed herein, wherein treatment is defined as the subject not developing a new BCC on the skin in a 12 month period once treatment has started.

The methods of treating Gorlin Syndrome in a subject in need thereof comprise topically administering to the face of the subject once daily an anhydrous rapamycin gel composition disclosed herein, wherein treatment is defined as the subject not developing a new BCC on the face in a 12 month period once treatment has started.

The methods of treating Gorlin Syndrome in a subject in need thereof comprise topically administering to the affected areas of the subject once daily an anhydrous rapamycin gel composition disclosed herein, wherein treatment is defined as the subject developing no more than 2 new BCCs on the skin in about a 6 month period once treatment has started.

The methods of treating Gorlin Syndrome in a subject in need thereof comprise topically administering to the affected areas of the subject once daily an anhydrous rapamycin gel composition disclosed herein, wherein treatment is defined as the subject developing no more than 2 new BCCs on the skin in about a 12 month period once treatment has started.

The methods of treating Gorlin Syndrome in a subject in need thereof comprise topically administering to the face of the subject once daily an anhydrous rapamycin gel composition disclosed herein, wherein treatment is defined as the subject developing no more than 2 new BCCs on the face in about a 12 month period once treatment has started.

The methods of treating Gorlin Syndrome in a subject in need thereof comprise topically administering to the affected areas of the subject once daily an anhydrous rapamycin gel composition disclosed herein, wherein treatment is defined as the subject developing no more than 1 new BCC on the skin or face in about a 6 month period once treatment has started.

The methods of treating Gorlin Syndrome in a subject in need thereof comprise topically administering to the affected areas of the subject once daily an anhydrous rapamycin gel composition disclosed herein, wherein treatment is defined as the subject developing no more than 1 new BCC on the skin in about a 12 month period once treatment has started.

The methods of treating Gorlin Syndrome in a subject in need thereof comprise topically administering to the face of the subject once daily an anhydrous rapamycin gel composition disclosed herein, wherein treatment is defined as the subject developing no more than 1 new BCC on the face in about a 12 month period once treatment has started.

The methods of treating Gorlin Syndrome in a subject in need thereof comprise topically administering to the affected areas of the subject once daily an anhydrous rapamycin gel composition disclosed herein, wherein treatment is defined as the size of the BCC lesion having decreased.

The methods of treating Gorlin Syndrome in a subject in need thereof comprise topically administering to the affected areas of the subject once daily an anhydrous rapamycin gel composition disclosed herein, wherein treatment is defined as the sum diameter of the longest BCC lesion on the skin having decreased.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the subject not developing a new BCC in a 6 month period once treatment has started.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the subject not developing a new BCC on the skin in a 12 month period once treatment has started.

The methods of decreasing the risk of progression or decreasing the risk of developing basal cell carcinoma in a subject with Gorlin Syndrome comprise topically administering to the face of the subject once daily an anhydrous rapamycin gel composition disclosed herein, wherein treatment is defined as the subject not developing a new BCC on the face in a 12 month period once treatment has started.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the subject developing no more than 2 new BCCs on the skin in about a 6 month period once treatment has started.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the subject developing no more than 2 new BCCs on the skin in about a 12 month period once treatment has started.

The methods of decreasing the risk of progression or decreasing the risk of developing basal cell carcinoma in a subject with Gorlin Syndrome comprise topically administering to the face of the subject once daily an anhydrous rapamycin gel composition disclosed herein, wherein treatment is defined as the subject developing no more than 2 new BCCs on the face in about a 12 month period once treatment has started.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the subject developing no more than 1 new BCC in about a 6 month period once treatment has started.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the subject developing no more than 1 new BCC on the skin in about a 12 month period once treatment has started.

The methods of decreasing the risk of progression or decreasing the risk of developing basal cell carcinoma in a subject with Gorlin Syndrome comprise topically administering to the face of the subject once daily an anhydrous rapamycin gel composition disclosed herein, wherein treatment is defined as the subject developing no more than 1 new BCC on the face in about a 12 month period once treatment has started.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the size of the BCC lesion having decreased.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as the sum diameter of the longest BCC lesion on the skin having decreased.

The methods of decreasing the risk of progression or decreasing the risk of developing basal cell carcinoma in a subject with Gorlin Syndrome comprise topically administering to the affected areas of the subject once daily an anhydrous rapamycin gel composition disclosed herein, wherein treatment is defined as a decrease in the small depressions in the palms of the hands and soles of the feet.

The subject of any of the aspects of the present invention may have one or more basal cell carcinomas (BCCs) on the skin, wherein the BCC is selected from the group consisting of Superficial, Nodular (solid), Micronodular, Infiltrative, Fibroepithelial (Pinkus tumor), Basal cell carcinoma with adnexal differentiation, Basosquamous carcinoma, Keratotic, or combinations thereof, and may have an anhydrous rapamycin gel composition disclosed herein topically administered to them in accordance with the present invention.

The subject of any of the aspects of the present invention may have one or more Superficial basal cell carcinomas (BCCs) on the skin and may have an anhydrous rapamycin gel composition disclosed herein topically administered to them in accordance with the present invention.

The subject of any of the aspects of the present invention may have one or more Nodular (solid) basal cell carcinomas (BCCs) on the skin and may have an anhydrous rapamycin gel composition disclosed herein topically administered to them in accordance with the present invention.

The subject of any of the aspects of the present invention may have one or more Micronodular basal cell carcinomas (BCCs) on the skin and may have an anhydrous rapamycin gel composition disclosed herein topically administered to them in accordance with the present invention.

The subject of any of the aspects of the present invention may have one or more Infiltrative basal cell carcinomas (BCCs) on the skin and may have an anhydrous rapamycin gel composition disclosed herein topically administered to them in accordance with the present invention.

The subject of any of the aspects of the present invention may have one or more Fibroepithelial (Pinkus tumor) basal cell carcinomas (BCCs) on the skin and may have an anhydrous rapamycin gel composition disclosed herein topically administered to them in accordance with the present invention.

The subject of any of the aspects of the present invention may have one or more basal cell carcinomas (BCCs) with adnexal differentiation on the skin and may have an anhydrous rapamycin gel composition disclosed herein topically administered to them in accordance with the present invention.

The subject of any of the aspects of the present invention may have one or more Basosquamous carcinoma on the skin and may have an anhydrous rapamycin gel composition disclosed herein topically administered to them in accordance with the present invention.

The subject of any of the aspects of the present invention may have one or more Keratotic basal cell carcinomas (BCCs) on the skin and may have an anhydrous rapamycin gel composition disclosed herein topically administered to them in accordance with the present invention.

The subject of any of the aspects of the present invention may see a decrease in surgical intervention after topically administering the anhydrous rapamycin gel composition disclosed herein in accordance with the present invention.

The subject of any of the aspects of the present invention may no longer require surgical intervention after topically administering the anhydrous rapamycin gel composition disclosed herein in accordance with the present invention.

The subject of any of the aspects of the present invention may develop no more than 2 new basal cell carcinomas (BCCs) on the skin in about a 12 month period after topically administering the anhydrous rapamycin gel composition disclosed herein in accordance with the present invention. The subject of any of the aspects of the present invention may develop no more than 2 new basal cell carcinomas (BCCs) on the skin in about a 11 month period after topically administering the anhydrous rapamycin gel composition disclosed herein in accordance with the present invention. The subject of any of the aspects of the present invention may develop no more than 2 new basal cell carcinomas (BCCs) on the skin in about a 10 month period after topically administering the anhydrous rapamycin gel composition disclosed herein in accordance with the present invention. The subject of any of the aspects of the present invention may develop no more than 2 new basal cell carcinomas (BCCs) on the skin in about a 9 month period after topically administering the anhydrous rapamycin gel composition disclosed herein in accordance with the present invention. The subject of any of the aspects of the present invention may develop no more than 2 new basal cell carcinomas (BCCs) on the skin in about a 8 month period after topically administering the anhydrous rapamycin gel composition disclosed herein in accordance with the present invention. The subject of any of the aspects of the present invention may develop no more than 2 new basal cell carcinomas (BCCs) on the skin in about a 7 month period after topically administering the anhydrous rapamycin gel composition disclosed herein in accordance with the present invention. The subject of any of the aspects of the present invention may develop no more than 2 new basal cell carcinomas (BCCs) on the skin in about a 6 month period after topically administering the anhydrous rapamycin gel composition disclosed herein in accordance with the present invention. The subject of any of the aspects of the present invention may develop no more than 2 new basal cell carcinomas (BCCs) on the skin in about a 5 month period after topically administering the anhydrous rapamycin gel composition disclosed herein in accordance with the present invention. The subject of any of the aspects of the present invention may develop no more than 2 new basal cell carcinomas (BCCs) on the skin in about a 4 month period after topically administering the anhydrous rapamycin gel composition disclosed herein in accordance with the present invention. The subject of any of the aspects of the present invention may develop no more than 2 new basal cell carcinomas (BCCs) on the skin in about a 3 month period after topically administering the anhydrous rapamycin gel composition disclosed herein in accordance with the present invention. The subject of any of the aspects of the present invention may develop no more than 2 new basal cell carcinomas (BCCs) on the skin in about a 2 month period after topically administering the anhydrous rapamycin gel composition disclosed herein in accordance with the present invention. The subject of any of the aspects of the present invention may develop no more than 2 new basal cell carcinomas (BCCs) on the skin in about a 1 month period after topically administering the anhydrous rapamycin gel composition disclosed herein in accordance with the present invention.

The subject of any of the aspects of the present invention may develop no more than 2 new basal cell carcinomas (BCCs) on the face in about a 12 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 11 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 10 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 9 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 8 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 7 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 6 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 5 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 4 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 3 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 2 month period after topically administering to the subject's face the anhydrous rapamycin gel composition disclosed herein in accordance with the present invention. The subject of any of the aspects of the present invention may develop no more than 2 new basal cell carcinomas (BCCs) on the face in about a 1 month period after topically administering to the subject's face the anhydrous rapamycin gel composition disclosed herein in accordance with the present invention.

The subject of any of the aspects of the present invention may develop no more than 1 new basal cell carcinoma (BCC) on the skin in about a 12 month period after topically administering the anhydrous rapamycin gel composition disclosed herein in accordance with the present invention. The subject of any of the aspects of the present invention may develop no more than 1 new basal cell carcinoma (BCC) on the skin in about a 11 month period after topically administering the anhydrous rapamycin gel composition disclosed herein in accordance with the present invention. The subject of any of the aspects of the present invention may develop no more than 1 new basal cell carcinoma (BCC) on the skin in about a 10 month period after topically administering the anhydrous rapamycin gel composition disclosed herein in accordance with the present invention. The subject of any of the aspects of the present invention may develop no more than 1 new basal cell carcinoma (BCC) on the skin in about a 9 month period after topically administering the anhydrous rapamycin gel composition disclosed herein in accordance with the present invention. The subject of any of the aspects of the present invention may develop no more than 1 new basal cell carcinoma (BCC) on the skin in about a 8 month period after topically administering the anhydrous rapamycin gel composition disclosed herein in accordance with the present invention. The subject of any of the aspects of the present invention may develop no more than 1 new basal cell carcinoma (BCC) on the skin in about a 7 month period after topically administering the anhydrous rapamycin gel composition disclosed herein in accordance with the present invention. The subject of any of the aspects of the present invention may develop no more than 1 new basal cell carcinoma (BCC) on the skin in about a 6 month period after topically administering the anhydrous rapamycin gel composition disclosed herein in accordance with the present invention. The subject of any of the aspects of the present invention may develop no more than 1 new basal cell carcinoma (BCC) on the skin in about a 5 month period after topically administering the anhydrous rapamycin gel composition disclosed herein in accordance with the present invention. The subject of any of the aspects of the present invention may develop no more than 1 new basal cell carcinoma (BCC) on the skin in about a 4 month period after topically administering the anhydrous rapamycin gel composition disclosed herein in accordance with the present invention. The subject of any of the aspects of the present invention may develop no more than 1 new basal cell carcinoma (BCC) on the skin in about a 3 month period after topically administering the anhydrous rapamycin gel composition disclosed herein in accordance with the present invention. The subject of any of the aspects of the present invention may develop no more than 1 new basal cell carcinoma (BCC) on the skin in about a 2 month period after topically administering the anhydrous rapamycin gel composition disclosed herein in accordance with the present invention. The subject of any of the aspects of the present invention may develop no more than 1 new basal cell carcinoma (BCC) on the skin in about a 1 month period after topically administering the anhydrous rapamycin gel composition disclosed herein in accordance with the present invention.

The subject of any of the aspects of the present invention may develop no more than 1 new basal cell carcinoma (BCC) on the face in about a 12 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 11 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 10 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 9 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 8 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 7 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 6 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 5 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 4 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 3 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 2 month period after topically administering to the subject's face the anhydrous rapamycin gel composition disclosed herein in accordance with the present invention. The subject of any of the aspects of the present invention may develop no more than 1 new basal cell carcinoma (BCC) on the face in about a 1 month period after topically administering to the subject's face the anhydrous rapamycin gel composition disclosed herein in accordance with the present invention.

The subject of any of the aspects of the present invention may develop no new basal cell carcinomas (BCCs) on the skin in about a 12 month period after topically administering the anhydrous rapamycin gel composition disclosed herein in accordance with the present invention. The subject of any of the aspects of the present invention may develop no new basal cell carcinomas (BCCs) on the skin in about a 11 month period after topically administering the anhydrous rapamycin gel composition disclosed herein in accordance with the present invention. The subject of any of the aspects of the present invention may develop no new basal cell carcinomas (BCCs) on the skin in about a 10 month period after topically administering the anhydrous rapamycin gel composition disclosed herein in accordance with the present invention. The subject of any of the aspects of the present invention may develop no new basal cell carcinomas (BCCs) on the skin in about a 9 month period after topically administering the anhydrous rapamycin gel composition disclosed herein in accordance with the present invention. The subject of any of the aspects of the present invention may develop no new basal cell carcinomas (BCCs) on the skin in about a 8 month period after topically administering the anhydrous rapamycin gel composition disclosed herein in accordance with the present invention. The subject of any of the aspects of the present invention may develop no new basal cell carcinomas (BCCs) on the skin in about a 7 month period after topically administering the anhydrous rapamycin gel composition disclosed herein in accordance with the present invention. The subject of any of the aspects of the present invention may develop no new basal cell carcinomas (BCCs) on the skin in about a 6 month period after topically administering the anhydrous rapamycin gel composition disclosed herein in accordance with the present invention. The subject of any of the aspects of the present invention may develop no new basal cell carcinomas (BCCs) on the skin in about a 5 month period after topically administering the anhydrous rapamycin gel composition disclosed herein in accordance with the present invention. The subject of any of the aspects of the present invention may develop no new basal cell carcinomas (BCCs) on the skin in about a 4 month period after topically administering the anhydrous rapamycin gel composition disclosed herein in accordance with the present invention. The subject of any of the aspects of the present invention may develop no new basal cell carcinomas (BCCs) on the skin in about a 3 month period after topically administering the anhydrous rapamycin gel composition disclosed herein in accordance with the present invention. The subject of any of the aspects of the present invention may develop no new basal cell carcinomas (BCCs) on the skin in about a 2 month period after topically administering the anhydrous rapamycin gel composition disclosed herein in accordance with the present invention. The subject of any of the aspects of the present invention may develop no new basal cell carcinomas (BCCs) on the skin in about a 1 month period after topically administering the anhydrous rapamycin gel composition disclosed herein in accordance with the present invention.

The subject of any of the aspects of the present invention may develop no new basal cell carcinomas (BCCs) on the face in about a 12 month period after topically administering to the subject's face the anhydrous rapamycin gel composition disclosed herein in accordance with the present invention. The subject of any of the aspects of the present invention may develop no new basal cell carcinomas (BCCs) on the face in about a 11 month period after topically administering to the subject's face the anhydrous rapamycin gel composition disclosed herein in accordance with the present invention. The subject of any of the aspects of the present invention may develop no new basal cell carcinomas (BCCs) on the face in about a 10 month period after topically administering to the subject's face the anhydrous rapamycin gel composition disclosed herein in accordance with the present invention. The subject of any of the aspects of the present invention may develop no new basal cell carcinomas (BCCs) on the face in about a 9 month period after topically administering to the subject's face the anhydrous rapamycin gel composition disclosed herein in accordance with the present invention. The subject of any of the aspects of the present invention may develop no new basal cell carcinomas (BCCs) on the face in about a 8 month period after topically administering to the subject's face the anhydrous rapamycin gel composition disclosed herein in accordance with the present invention. The subject of any of the aspects of the present invention may develop no new basal cell carcinomas (BCCs) on the face in about a 7 month period after topically administering to the subject's face the anhydrous rapamycin gel composition disclosed herein in accordance with the present invention. The subject of any of the aspects of the present invention may develop no new basal cell carcinomas (BCCs) on the face in about a 6 month period after topically administering to the subject's face the anhydrous rapamycin gel composition disclosed herein in accordance with the present invention. The subject of any of the aspects of the present invention may develop no new basal cell carcinomas (BCCs) on the face in about a 5 month period after topically administering to the subject's face the anhydrous rapamycin gel composition disclosed herein in accordance with the present invention. The subject of any of the aspects of the present invention may develop no new basal cell carcinomas (BCCs) on the face in about a 4 month period after topically administering to the subject's face the anhydrous rapamycin gel composition disclosed herein in accordance with the present invention. The subject of any of the aspects of the present invention may develop no new basal cell carcinomas (BCCs) on the face in about a 3 month period after topically administering to the subject's face the anhydrous rapamycin gel composition disclosed herein in accordance with the present invention. The subject of any of the aspects of the present invention may develop no new basal cell carcinomas (BCCs) on the s face in about a 2 month period after topically administering to the subject's face the anhydrous rapamycin gel composition disclosed herein in accordance with the present invention. The subject of any of the aspects of the present invention may develop no new basal cell carcinomas (BCCs) on the face in about a 1 month period after topically administering to the subject's face the anhydrous rapamycin gel composition disclosed herein in accordance with the present invention.

The BCC of any of the aspects of the present invention may be selected from the group consisting of Superficial, Nodular (solid), Micronodular, Infiltrative, Fibroepithelial (Pinkus tumor), Basal cell carcinoma with adnexal differentiation, Basosquamous carcinoma, Keratotic, or combinations thereof.

The lesion size of a basal cell carcinoma (BCC) on the skin of the subject of any of the aspects of the present invention may have decreased in size after topically administering the anhydrous rapamycin gel composition disclosed herein in accordance with the present invention.

The sum diameter of the longest basal cell carcinoma (BCC) lesion on the skin of the subject of any of the aspects of the present invention may have decreased in size after topically administering the anhydrous rapamycin gel composition disclosed herein in accordance with the present invention.

The subject of any of the aspects of the present invention may see an increase in the time until a new basal cell carcinoma (BCC) biopsy is confirmed after topically administering the anhydrous rapamycin gel composition disclosed herein in accordance with the present invention. The subject of any of the aspects of the present invention may see an increase in about a month until a new basal cell carcinoma (BCC) biopsy is confirmed after topically administering the anhydrous rapamycin gel composition disclosed herein in accordance with the present invention. The subject of any of the aspects of the present invention may see an increase in about 2 months until a new basal cell carcinoma (BCC) biopsy is confirmed after topically administering the anhydrous rapamycin gel composition disclosed herein in accordance with the present invention. The subject of any of the aspects of the present invention may see an increase in about 3 months until a new basal cell carcinoma (BCC) biopsy is confirmed after topically administering the anhydrous rapamycin gel composition disclosed herein in accordance with the present invention. The subject of any of the aspects of the present invention may see an increase in about 4 months until a new basal cell carcinoma (BCC) biopsy is confirmed after topically administering the anhydrous rapamycin gel composition disclosed herein in accordance with the present invention. The subject of any of the aspects of the present invention may see an increase in about 5 months until a new basal cell carcinoma (BCC) biopsy is confirmed after topically administering the anhydrous rapamycin gel composition disclosed herein in accordance with the present invention. The subject of any of the aspects of the present invention may see an increase in about 6 months until a new basal cell carcinoma (BCC) biopsy is confirmed after topically administering the anhydrous rapamycin gel composition disclosed herein in accordance with the present invention. The subject of any of the aspects of the present invention may see an increase in about 7 months until a new basal cell carcinoma (BCC) biopsy is confirmed after topically administering the anhydrous rapamycin gel composition disclosed herein in accordance with the present invention. The subject of any of the aspects of the present invention may see an increase in about 8 months until a new basal cell carcinoma (BCC) biopsy is confirmed after topically administering the anhydrous rapamycin gel composition disclosed herein in accordance with the present invention. The subject of any of the aspects of the present invention may see an increase in about 9 months until a new basal cell carcinoma (BCC) biopsy is confirmed after topically administering the anhydrous rapamycin gel composition disclosed herein in accordance with the present invention. The subject of any of the aspects of the present invention may see an increase in about 10 months until a new basal cell carcinoma (BCC) biopsy is confirmed after topically administering the anhydrous rapamycin gel composition disclosed herein in accordance with the present invention. The subject of any of the aspects of the present invention may see an increase in about 11 months until a new basal cell carcinoma (BCC) biopsy is confirmed after topically administering the anhydrous rapamycin gel composition disclosed herein in accordance with the present invention. The subject of any of the aspects of the present invention may see an increase in about 12 months until a new basal cell carcinoma (BCC) biopsy is confirmed after topically administering the anhydrous rapamycin gel composition disclosed herein in accordance with the present invention. Preferably, the time to identify a new confirmed BCC biopsy is greater than about 1 month.

The subject of any of the aspects of the present invention may see improvement in the score on the advanced basal cell carcinoma index (aBCCdex) after topically administering the anhydrous rapamycin gel composition disclosed herein in accordance with the present invention. The subject of any of the aspects of the present invention may see an improvement of at least 2 points in the score on the aBCCdex after topically administering the anhydrous rapamycin gel composition disclosed herein in accordance with the present invention. The subject of any of the aspects of the present invention may see an improvement of at least 3 points in the score on the aBCCdex after topically administering the anhydrous rapamycin gel composition disclosed herein in accordance with the present invention. The subject of any of the aspects of the present invention may see an improvement of at least 4 points in the score on the aBCCdex after topically administering the anhydrous rapamycin gel composition disclosed herein in accordance with the present invention. The subject of any of the aspects of the present invention may see an improvement of at least 5 points in the score on the aBCCdex after topically administering the anhydrous rapamycin gel composition disclosed herein in accordance with the present invention. The subject of any of the aspects of the present invention may see an improvement of at least 6 points in the score on the aBCCdex after topically administering the anhydrous rapamycin gel composition disclosed herein in accordance with the present invention. The subject of any of the aspects of the present invention may see an improvement of at least 7 points in the score on the aBCCdex after topically administering the anhydrous rapamycin gel composition disclosed herein in accordance with the present invention. The subject of any of the aspects of the present invention may see an improvement of at least 8 points in the score on the aBCCdex after topically administering the anhydrous rapamycin gel composition disclosed herein in accordance with the present invention. The subject of any of the aspects of the present invention may see an improvement of at least 9 points in the score on the aBCCdex after topically administering the anhydrous rapamycin gel composition disclosed herein in accordance with the present invention. The subject of any of the aspects of the present invention may see an improvement of at least 10 points in the score on the aBCCdex after topically administering the anhydrous rapamycin gel composition disclosed herein in accordance with the present invention. The subject of any of the aspects of the present invention may see an improvement of at least 15 points in the score on the aBCCdex after topically administering the anhydrous rapamycin gel composition disclosed herein in accordance with the present invention. The subject of any of the aspects of the present invention may see an improvement of at least 20 points in the score on the aBCCdex after topically administering the anhydrous rapamycin gel composition disclosed herein in accordance with the present invention. The subject of any of the aspects of the present invention may see an improvement of at least 25 points in the score on the aBCCdex after topically administering the anhydrous rapamycin gel composition disclosed herein in accordance with the present invention. The subject of any of the aspects of the present invention may see an improvement of at least 30 points in the score on the aBCCdex after topically administering the anhydrous rapamycin gel composition disclosed herein in accordance with the present invention.

The subject of any of the aspects of the present invention may see an improvement of at least 1 point in the score on one or more questions of the aBCCdex after topically administering the anhydrous rapamycin gel composition disclosed herein in accordance with the present invention. The subject of any of the aspects of the present invention may see an improvement of at least 2 points in the score on one or more questions of the aBCCdex after topically administering the anhydrous rapamycin gel composition disclosed herein in accordance with the present invention. The subject of any of the aspects of the present invention may see an improvement of at least 3 points in the score on one or more questions of the aBCCdex after topically administering the anhydrous rapamycin gel composition disclosed herein in accordance with the present invention. The subject of any of the aspects of the present invention may see an improvement of at least 4 points in the score on one or more questions of the aBCCdex after topically administering the anhydrous rapamycin gel composition disclosed herein in accordance with the present invention. The subject of any of the aspects of the present invention may see an improvement of at least 5 points in the score on one or more questions of the aBCCdex after topically administering the anhydrous rapamycin gel composition disclosed herein in accordance with the present invention. The subject of any of the aspects of the present invention may see an improvement of at least 6 points in the score on one or more questions of the aBCCdex after topically administering the anhydrous rapamycin gel composition disclosed herein in accordance with the present invention.

The subject of any of the aspects of the present invention may see improvement in the score on the dermatology life quality index (DLQI) after topically administering the anhydrous rapamycin gel composition disclosed herein in accordance with the present invention. The subject of any of the aspects of the present invention may see an improvement of at least 2 points in the score on the DLQI after topically administering the anhydrous rapamycin gel composition disclosed herein in accordance with the present invention. The subject of any of the aspects of the present invention may see an improvement of at least 3 points in the score on the DLQI after topically administering the anhydrous rapamycin gel composition disclosed herein in accordance with the present invention. The subject of any of the aspects of the present invention may see an improvement of at least 4 points in the score on the DLQI after topically administering the anhydrous rapamycin gel composition disclosed herein in accordance with the present invention. The subject of any of the aspects of the present invention may see an improvement of at least 5 points in the score on the DLQI after topically administering the anhydrous rapamycin gel composition disclosed herein in accordance with the present invention. The subject of any of the aspects of the present invention may see an improvement of at least 6 points in the score on the DLQI after topically administering the anhydrous rapamycin gel composition disclosed herein in accordance with the present invention. The subject of any of the aspects of the present invention may see an improvement of at least 7 points in the score on the DLQI after topically administering the anhydrous rapamycin gel composition disclosed herein in accordance with the present invention. The subject of any of the aspects of the present invention may see an improvement of at least 8 points in the score on the DLQI after topically administering the anhydrous rapamycin gel composition disclosed herein in accordance with the present invention. The subject of any of the aspects of the present invention may see an improvement of at least 9 points in the score on the DLQI after topically administering the anhydrous rapamycin gel composition disclosed herein in accordance with the present invention. The subject of any of the aspects of the present invention may see an improvement of at least 10 points in the score on the DLQI after topically administering the anhydrous rapamycin gel composition disclosed herein in accordance with the present invention. The subject of any of the aspects of the present invention may see an improvement of at least 15 points in the score on the DLQI after topically administering the anhydrous rapamycin gel composition disclosed herein in accordance with the present invention. The subject of any of the aspects of the present invention may see an improvement of at least 20 points in the score on the DLQI after topically administering the anhydrous rapamycin gel composition disclosed herein in accordance with the present invention. The subject of any of the aspects of the present invention may see an improvement of at least 25 points in the score on the DLQI after topically administering the anhydrous rapamycin gel composition disclosed herein in accordance with the present invention. The subject of any of the aspects of the present invention may see an improvement of at least 30 points in the score on the DLQI after topically administering the anhydrous rapamycin gel composition disclosed herein in accordance with the present invention.

The subject of any of the aspects of the present invention may see an improvement of at least 1 point in the score on one or more questions of the DLQI after topically administering the anhydrous rapamycin gel composition disclosed herein in accordance with the present invention. The subject of any of the aspects of the present invention may see an improvement of at least 2 points in the score on one or more questions of the DLQI after topically administering the anhydrous rapamycin gel composition disclosed herein in accordance with the present invention.

### Anhydrous Rapamycin Gel Compositions Administered Herein

In some embodiments, the composition is an anhydrous rapamycin gel composition comprising rapamycin present from about 0.1 wt % to about 5 wt % of the total composition, one or more solvents present from about 80 wt % to about 99 wt % of the total composition, a gelling agent present from about 0.1 wt % to about 5 wt % of the total composition, an antioxidant is present from about 0.001 wt % to about 1 wt % of the total composition, and, optionally a buffer.

### Rapamycin—Active Ingredient

In some embodiments, the anhydrous rapamycin gel composition disclosed herein comprises rapamycin present in an amount of about 0.1% to about 5% of the total weight of the composition. In some embodiments, the anhydrous rapamycin gel composition disclosed herein comprises rapamycin present in an amount of about 0.1% to about 4.5% of the total weight of the composition. In some embodiments, the anhydrous rapamycin gel composition disclosed herein comprises rapamycin present in an amount of about 0.1% to about 4% of the total weight of the composition. In some embodiments, the anhydrous rapamycin gel composition disclosed herein comprises rapamycin present in an amount of about 0.1% to about 3.5% of the total weight of the composition. In some embodiments, the anhydrous rapamycin gel composition disclosed herein comprises rapamycin present in an amount of about 0.1% to about 3% of the total weight of the composition. In some embodiments, the anhydrous rapamycin gel composition disclosed herein comprises rapamycin present in an amount of about 0.1% to about 2.5% of the total weight of the composition. In some embodiments, the anhydrous rapamycin gel composition disclosed herein comprises rapamycin present in an amount of about 0.1% to about 2% of the total weight of the composition. In some embodiments, the anhydrous rapamycin gel composition disclosed herein comprises rapamycin present in an amount of about 0.1% to about 1.5% of the total weight of the composition. In some embodiments, the anhydrous rapamycin gel composition disclosed herein comprises rapamycin present in an amount of about 0.1% to about 1% of the total weight of the composition. In some embodiments, the anhydrous rapamycin gel composition disclosed herein comprises rapamycin present in an amount of about 1% to about 5% of the total weight of the composition. In some embodiments, the anhydrous rapamycin gel composition disclosed herein comprises rapamycin present in an amount of about 1.5% to about 5% of the total weight of the composition. In some embodiments, the anhydrous rapamycin gel composition disclosed herein comprises rapamycin present in an amount of about 2% to about 5% of the total weight of the composition. In some embodiments, the anhydrous rapamycin gel composition disclosed herein comprises rapamycin present in an amount of about 2.5% to about 5% of the total weight of the composition. In some embodiments, the anhydrous rapamycin gel composition disclosed herein comprises rapamycin present in an amount of about 3% to about 5% of the total weight of the composition. In some embodiments, the anhydrous rapamycin gel composition disclosed herein comprises rapamycin present in an amount of about 3.5% to about 5% of the total weight of the composition. In some embodiments, the anhydrous rapamycin gel composition disclosed herein comprises rapamycin present in an amount of about 4% to about 5% of the total weight of the composition. In some embodiments, the anhydrous rapamycin gel composition disclosed herein comprises rapamycin present in an amount of about 4.5% to about 5% of the total weight of the composition. Preferably, the anhydrous rapamycin gel composition disclosed herein comprises rapamycin present in an amount of about 3.9% of the total weight of the composition.

### Solvent System

In some embodiments, the anhydrous rapamycin gel composition disclosed herein comprises one or more solvent present in an amount of about 80% to about 99% of the total weight of the composition. In some embodiments, the anhydrous rapamycin gel composition disclosed herein comprises one or more solvent present in an amount of about 80% to about 98% of the total weight of the composition. In some embodiments, the anhydrous rapamycin gel composition disclosed herein comprises one or more solvent present in an amount of about 80% to about 97% of the total weight of the composition. In some embodiments, the anhydrous rapamycin gel composition disclosed herein comprises one or more solvent present in an amount of about 80% to about 96% of the total weight of the composition. In some embodiments, the anhydrous rapamycin gel composition disclosed herein comprises one or more solvent present in an amount of about 80% to about 95% of the total weight of the composition. In some embodiments, the anhydrous rapamycin gel composition disclosed herein comprises one or more solvent present in an amount of about 80% to about 90% of the total weight of the composition. In some embodiments, the anhydrous rapamycin gel composition disclosed herein comprises one or more solvent present in an amount of about 80% to about 85% of the total weight of the composition. Preferably, the anhydrous rapamycin gel composition disclosed herein comprises one or more solvent present in an amount of about 95% of the total weight of the composition. More preferably, the anhydrous rapamycin gel composition disclosed herein comprises four solvents present in a total amount of about 94.9% of the total weight of the composition.

In some embodiments, one or more solvents are selected from propylene glycol, benzyl alcohol, DMSO, diglycol, propylene glycol monocaprylate, diethylene glycol monoethylether, tetrahydrofurfurylalcohol polyethylene glycol ether, butylene glycol, diethylene glycol, triethylene glycol, polyethylene glycol, diisopropyl adipate, isopropyl alcohol, glycerol, and combinations thereof. Preferably, the anhydrous rapamycin gel composition disclosed herein comprises four solvents comprising isopropyl alcohol, polyethylene glycol 400, diisopropyl adipate, and glycerol.

In some embodiments, the anhydrous rapamycin gel composition disclosed herein comprises isopropyl alcohol as the one or more solvent in an amount of about 5% to about 55% of the total weight of the composition. In some embodiments, the anhydrous rapamycin gel composition comprises isopropyl alcohol as the one or more solvent in an amount of about 5% to about 50% of the total weight of the composition. In some embodiments, the anhydrous rapamycin gel composition disclosed herein comprises isopropyl alcohol as the one or more solvent in an amount of about 5% to about 45% of the total weight of the composition. In some embodiments, the anhydrous rapamycin gel composition disclosed herein comprises isopropyl alcohol as the one or more solvent in an amount of about 5% to about 40% of the total weight of the composition. In some embodiments, the anhydrous rapamycin gel composition disclosed herein comprises isopropyl alcohol as the one or more solvent in an amount of about 5% to about 35% of the total weight of the composition. In some embodiments, the anhydrous rapamycin gel composition disclosed herein comprises isopropyl alcohol as the one or more solvent in an amount of about 5% to about 30% of the total weight of the composition. In some embodiments, the anhydrous rapamycin gel composition disclosed herein comprises isopropyl alcohol as the one or more solvent in an amount of about 5% to about 25% of the total weight of the composition. In some embodiments, the anhydrous rapamycin gel composition disclosed herein comprises isopropyl alcohol as the one or more solvent in an amount of about 5% to about 20% of the total weight of the composition. In some embodiments, the anhydrous rapamycin gel composition disclosed herein comprises isopropyl alcohol as the one or more solvent in an amount of about 5% to about 15% of the total weight of the composition. In some embodiments, the anhydrous rapamycin gel composition disclosed herein comprises isopropyl alcohol as the one or more solvent in an amount of about 5% to about 10% of the total weight of the composition. Preferably, the anhydrous rapamycin gel composition disclosed herein comprises isopropyl alcohol as the one or more solvent in an amount of about 15% of the total weight of the composition.

In some embodiments, the anhydrous rapamycin gel composition disclosed herein comprises polyethylene glycol as the one or more solvent in an amount of about 5% to about 55% of the total weight of the composition. In some embodiments, the anhydrous rapamycin gel composition disclosed herein comprises polyethylene glycol as the one or more solvent in an amount of about 5% to about 50% of the total weight of the composition. In some embodiments, the anhydrous rapamycin gel composition disclosed herein comprises polyethylene glycol as the one or more solvent in an amount of about 5% to about 45% of the total weight of the composition. In some embodiments, the anhydrous rapamycin gel composition disclosed herein comprises polyethylene glycol as the one or more solvent in an amount of about 5% to about 40% of the total weight of the composition. In some embodiments, the anhydrous rapamycin gel composition disclosed herein comprises polyethylene glycol as the one or more solvent in an amount of about 5% to about 35% of the total weight of the composition. In some embodiments, the anhydrous rapamycin gel composition disclosed herein comprises polyethylene glycol as the one or more solvent in an amount of about 5% to about 30% of the total weight of the composition. In some embodiments, the anhydrous rapamycin gel composition disclosed herein comprises polyethylene glycol as the one or more solvent in an amount of about 5% to about 25% of the total weight of the composition. In some embodiments, the anhydrous rapamycin gel composition disclosed herein comprises polyethylene glycol as the one or more solvent in an amount of about 5% to about 20% of the total weight of the composition. In some embodiments, the anhydrous rapamycin gel composition disclosed herein comprises polyethylene glycol as the one or more solvent in an amount of about 5% to about 15% of the total weight of the composition. In some embodiments, the anhydrous rapamycin gel composition disclosed herein comprises polyethylene glycol as the one or more solvent in an amount of about 5% to about 10% of the total weight of the composition. Preferably, the anhydrous rapamycin gel composition disclosed herein comprises polyethylene glycol 400 as the one or more solvent in an amount of about 54.9% of the total weight of the composition.

In some embodiments, the anhydrous rapamycin gel composition disclosed herein comprises diisopropyl adipate as the one or more solvent in an amount of about 5% to about 55% of the total weight of the composition. In some embodiments, the anhydrous rapamycin gel composition disclosed herein comprises diisopropyl adipate as the one or more solvent in an amount of about 5% to about 50% of the total weight of the composition. In some embodiments, the anhydrous rapamycin gel composition disclosed herein comprises diisopropyl adipate as the one or more solvent in an amount of about 5% to about 45% of the total weight of the composition. In some embodiments, the anhydrous rapamycin gel composition disclosed herein comprises diisopropyl adipate as the one or more solvent in an amount of about 5% to about 40% of the total weight of the composition. In some embodiments, the anhydrous rapamycin gel composition disclosed herein comprises diisopropyl adipate as the one or more solvent in an amount of about 5% to about 35% of the total weight of the composition. In some embodiments, the anhydrous rapamycin gel composition disclosed herein comprises diisopropyl adipate as the one or more solvent in an amount of about 5% to about 30% of the total weight of the composition. In some embodiments, the anhydrous rapamycin gel composition disclosed herein comprises diisopropyl adipate as the one or more solvent in an amount of about 5% to about 25% of the total weight of the composition. In some embodiments, the anhydrous rapamycin gel composition disclosed herein comprises diisopropyl adipate as the one or more solvent in an amount of about 5% to about 20% of the total weight of the composition. In some embodiments, the anhydrous rapamycin gel composition disclosed herein comprises diisopropyl adipate as the one or more solvent in an amount of about 5% to about 15% of the total weight of the composition. In some embodiments, the anhydrous rapamycin gel composition disclosed herein comprises diisopropyl adipate as the one or more solvent in an amount of about 5% to about 10% of the total weight of the composition. Preferably, the anhydrous rapamycin gel composition disclosed herein comprises diisopropyl adipate as the one or more solvent in an amount of about 15% of the total weight of the composition.

In some embodiments, the anhydrous rapamycin gel composition disclosed herein comprises glycerol as the one or more solvent in an amount of about 5% to about 55% of the total weight of the composition. In some embodiments, the anhydrous rapamycin gel composition disclosed herein comprises glycerol as the one or more solvent in an amount of about 5% to about 50% of the total weight of the composition. In some embodiments, the anhydrous rapamycin gel composition disclosed herein comprises glycerol as the one or more solvent in an amount of about 5% to about 45% of the total weight of the composition. In some embodiments, the anhydrous rapamycin gel composition disclosed herein comprises glycerol as the one or more solvent in an amount of about 5% to about 40% of the total weight of the composition. In some embodiments, the anhydrous rapamycin gel composition disclosed herein comprises glycerol as the one or more solvent in an amount of about 5% to about 35% of the total weight of the composition. In some embodiments, the anhydrous rapamycin gel composition disclosed herein comprises glycerol as the one or more solvent in an amount of about 5% to about 30% of the total weight of the composition. In some embodiments, the anhydrous rapamycin gel composition disclosed herein comprises glycerol as the one or more solvent in an amount of about 5% to about 25% of the total weight of the composition. In some embodiments, the anhydrous rapamycin gel composition disclosed herein comprises glycerol as the one or more solvent in an amount of about 5% to about 20% of the total weight of the composition. In some embodiments, the anhydrous rapamycin gel composition disclosed herein comprises glycerol as the one or more solvent in an amount of about 5% to about 15% of the total weight of the composition. In some embodiments, the anhydrous rapamycin gel composition disclosed herein comprises glycerol as the one or more solvent in an amount of about 5% to about 10% of the total weight of the composition. Preferably, the anhydrous rapamycin gel composition disclosed herein comprises glycerol as the one or more solvent in an amount of about 10% of the total weight of the composition.

In some embodiments, the anhydrous rapamycin gel composition disclosed herein comprises propylene glycol as the one or more solvent in an amount of about 5% to about 55% of the total weight of the composition. In some embodiments, the anhydrous rapamycin gel composition disclosed herein comprises propylene glycol as the one or more solvent in an amount of about 5% to about 50% of the total weight of the composition. In some embodiments, the anhydrous rapamycin gel composition disclosed herein comprises propylene glycol as the one or more solvent in an amount of about 5% to about 45% of the total weight of the composition. In some embodiments, the anhydrous rapamycin gel composition disclosed herein comprises propylene glycol as the one or more solvent in an amount of about 5% to about 40% of the total weight of the composition. In some embodiments, the anhydrous rapamycin gel composition disclosed herein comprises propylene glycol as the one or more solvent in an amount of about 5% to about 35% of the total weight of the composition. In some embodiments, the anhydrous rapamycin gel composition disclosed herein comprises propylene glycol as the one or more solvent in an amount of about 5% to about 30% of the total weight of the composition. In some embodiments, the anhydrous rapamycin gel composition disclosed herein comprises propylene glycol as the one or more solvent in an amount of about 5% to about 25% of the total weight of the composition. In some embodiments, the anhydrous rapamycin gel composition disclosed herein comprises propylene glycol as the one or more solvent in an amount of about 5% to about 20% of the total weight of the composition. In some embodiments, the anhydrous rapamycin gel composition disclosed herein comprises propylene glycol as the one or more solvent in an amount of about 5% to about 15% of the total weight of the composition. In some embodiments, the anhydrous rapamycin gel composition disclosed herein comprises propylene glycol as the one or more solvent in an amount of about 5% to about 10% of the total weight of the composition.

In some embodiments, the anhydrous rapamycin gel composition disclosed herein comprises benzyl alcohol as the one or more solvent in an amount of about 5% to about 55% of the total weight of the composition. In some embodiments, the anhydrous rapamycin gel composition disclosed herein comprises benzyl alcohol as the one or more solvent in an amount of about 5% to about 50% of the total weight of the composition. In some embodiments, the anhydrous rapamycin gel composition disclosed herein comprises benzyl alcohol as the one or more solvent in an amount of about 5% to about 45% of the total weight of the composition. In some embodiments, the anhydrous rapamycin gel composition disclosed herein comprises benzyl alcohol as the one or more solvent in an amount of about 5% to about 40% of the total weight of the composition. In some embodiments, the anhydrous rapamycin gel composition disclosed herein comprises benzyl alcohol as the one or more solvent in an amount of about 5% to about 35% of the total weight of the composition. In some embodiments, the anhydrous rapamycin gel composition disclosed herein comprises benzyl alcohol as the one or more solvent in an amount of about 5% to about 30% of the total weight of the composition. In some embodiments, the anhydrous rapamycin gel composition disclosed herein comprises benzyl alcohol as the one or more solvent in an amount of about 5% to about 25% of the total weight of the composition. In some embodiments, the anhydrous rapamycin gel composition disclosed herein comprises benzyl alcohol as the one or more solvent in an amount of about 5% to about 20% of the total weight of the composition. In some embodiments, the anhydrous rapamycin gel composition disclosed herein comprises benzyl alcohol as the one or more solvent in an amount of about 5% to about 15% of the total weight of the composition. In some embodiments, the anhydrous rapamycin gel composition disclosed herein comprises benzyl alcohol as the one or more solvent in an amount of about 5% to about 10% of the total weight of the composition.

In some embodiments, the anhydrous rapamycin gel composition disclosed herein comprises DMSO as the one or more solvent in an amount of about 5% to about 55% of the total weight of the composition. In some embodiments, the anhydrous rapamycin gel composition disclosed herein comprises DMSO as the one or more solvent in an amount of about 5% to about 50% of the total weight of the composition. In some embodiments, the anhydrous rapamycin gel composition disclosed herein comprises DMSO as the one or more solvent in an amount of about 5% to about 45% of the total weight of the composition. In some embodiments, the anhydrous rapamycin gel composition disclosed herein comprises DMSO as the one or more solvent in an amount of about 5% to about 40% of the total weight of the composition. In some embodiments, the anhydrous rapamycin gel composition disclosed herein comprises DMSO as the one or more solvent in an amount of about 5% to about 35% of the total weight of the composition. In some embodiments, the anhydrous rapamycin gel composition disclosed herein comprises DMSO as the one or more solvent in an amount of about 5% to about 30% of the total weight of the composition. In some embodiments, the anhydrous rapamycin gel composition disclosed herein comprises DMSO as the one or more solvent in an amount of about 5% to about 25% of the total weight of the composition. In some embodiments, the anhydrous rapamycin gel composition disclosed herein comprises DMSO as the one or more solvent in an amount of about 5% to about 20% of the total weight of the composition. In some embodiments, the anhydrous rapamycin gel composition disclosed herein comprises DMSO as the one or more solvent in an amount of about 5% to about 15% of the total weight of the composition. In some embodiments, the anhydrous rapamycin gel composition disclosed herein comprises DMSO as the one or more solvent in an amount of about 5% to about 10% of the total weight of the composition.

In some embodiments, the anhydrous rapamycin gel composition disclosed herein comprises diglycol as the one or more solvent in an amount of about 5% to about 55% of the total weight of the composition. In some embodiments, the anhydrous rapamycin gel composition disclosed herein comprises diglycol as the one or more solvent in an amount of about 5% to about 50% of the total weight of the composition. In some embodiments, the anhydrous rapamycin gel composition disclosed herein comprises diglycol as the one or more solvent in an amount of about 5% to about 45% of the total weight of the composition. In some embodiments, the anhydrous rapamycin gel composition disclosed herein comprises diglycol as the one or more solvent in an amount of about 5% to about 40% of the total weight of the composition. In some embodiments, the anhydrous rapamycin gel composition disclosed herein comprises diglycol as the one or more solvent in an amount of about 5% to about 35% of the total weight of the composition. In some embodiments, the anhydrous rapamycin gel composition disclosed herein comprises diglycol as the one or more solvent in an amount of about 5% to about 30% of the total weight of the composition. In some embodiments, the anhydrous rapamycin gel composition disclosed herein comprises diglycol as the one or more solvent in an amount of about 5% to about 25% of the total weight of the composition. In some embodiments, the anhydrous rapamycin gel composition disclosed herein comprises diglycol as the one or more solvent in an amount of about 5% to about 20% of the total weight of the composition. In some embodiments, the anhydrous rapamycin gel composition disclosed herein comprises diglycol as the one or more solvent in an amount of about 5% to about 15% of the total weight of the composition. In some embodiments, the anhydrous rapamycin gel composition disclosed herein comprises diglycol as the one or more solvent in an amount of about 5% to about 10% of the total weight of the composition.

In some embodiments, the anhydrous rapamycin gel composition disclosed herein comprises propylene glycol monocaprylate as the one or more solvent in an amount of about 5% to about 55% of the total weight of the composition. In some embodiments, the anhydrous rapamycin gel composition disclosed herein comprises propylene glycol monocaprylate as the one or more solvent in an amount of about 5% to about 50% of the total weight of the composition. In some embodiments, the anhydrous rapamycin gel composition disclosed herein comprises propylene glycol monocaprylate as the one or more solvent in an amount of about 5% to about 45% of the total weight of the composition. In some embodiments, the anhydrous rapamycin gel composition disclosed herein comprises propylene glycol monocaprylate as the one or more solvent in an amount of about 5% to about 40% of the total weight of the composition. In some embodiments, the anhydrous rapamycin gel composition disclosed herein comprises propylene glycol monocaprylate as the one or more solvent in an amount of about 5% to about 35% of the total weight of the composition. In some embodiments, the anhydrous rapamycin gel composition disclosed herein comprises propylene glycol monocaprylate as the one or more solvent in an amount of about 5% to about 30% of the total weight of the composition. In some embodiments, the anhydrous rapamycin gel composition disclosed herein comprises propylene glycol monocaprylate as the one or more solvent in an amount of about 5% to about 25% of the total weight of the composition. In some embodiments, the anhydrous rapamycin gel composition disclosed herein comprises propylene glycol monocaprylate as the one or more solvent in an amount of about 5% to about 20% of the total weight of the composition. In some embodiments, the anhydrous rapamycin gel composition disclosed herein comprises propylene glycol monocaprylate as the one or more solvent in an amount of about 5% to about 15% of the total weight of the composition. In some embodiments, the anhydrous rapamycin gel composition disclosed herein comprises propylene glycol monocaprylate as the one or more solvent in an amount of about 5% to about 10% of the total weight of the composition.

In some embodiments, the anhydrous rapamycin gel composition disclosed herein comprises diethylene glycol monoethylether as the one or more solvent in an amount of about 5% to about 55% of the total weight of the composition. In some embodiments, the anhydrous rapamycin gel composition disclosed herein comprises diethylene glycol monoethylether as the one or more solvent in an amount of about 5% to about 50% of the total weight of the composition. In some embodiments, the anhydrous rapamycin gel composition disclosed herein comprises diethylene glycol monoethylether as the one or more solvent in an amount of about 5% to about 45% of the total weight of the composition. In some embodiments, the anhydrous rapamycin gel composition disclosed herein comprises diethylene glycol monoethylether as the one or more solvent in an amount of about 5% to about 40% of the total weight of the composition. In some embodiments, the anhydrous rapamycin gel composition disclosed herein comprises diethylene glycol monoethylether as the one or more solvent in an amount of about 5% to about 35% of the total weight of the composition. In some embodiments, the anhydrous rapamycin gel composition disclosed herein comprises diethylene glycol monoethylether as the one or more solvent in an amount of about 5% to about 30% of the total weight of the composition. In some embodiments, the anhydrous rapamycin gel composition disclosed herein comprises diethylene glycol monoethylether as the one or more solvent in an amount of about 5% to about 25% of the total weight of the composition. In some embodiments, the anhydrous rapamycin gel composition disclosed herein comprises diethylene glycol monoethylether as the one or more solvent in an amount of about 5% to about 20% of the total weight of the composition. In some embodiments, the anhydrous rapamycin gel composition disclosed herein comprises diethylene glycol monoethylether as the one or more solvent in an amount of about 5% to about 15% of the total weight of the composition. In some embodiments, the anhydrous rapamycin gel composition disclosed herein comprises diethylene glycol monoethylether as the one or more solvent in an amount of about 5% to about 10% of the total weight of the composition.

In some embodiments, the anhydrous rapamycin gel composition disclosed herein comprises tetrahydrofurfurylalcohol polyethylene glycol ether as the one or more solvent in an amount of about 5% to about 55% of the total weight of the composition. In some embodiments, the anhydrous rapamycin gel composition disclosed herein comprises tetrahydrofurfurylalcohol polyethylene glycol ether as the one or more solvent in an amount of about 5% to about 50% of the total weight of the composition. In some embodiments, the anhydrous rapamycin gel composition disclosed herein comprises tetrahydrofurfurylalcohol polyethylene glycol ether as the one or more solvent in an amount of about 5% to about 45% of the total weight of the composition. In some embodiments, the anhydrous rapamycin gel composition disclosed herein comprises tetrahydrofurfurylalcohol polyethylene glycol ether as the one or more solvent in an amount of about 5% to about 40% of the total weight of the composition. In some embodiments, the anhydrous rapamycin gel composition disclosed herein comprises tetrahydrofurfurylalcohol polyethylene glycol ether as the one or more solvent in an amount of about 5% to about 35% of the total weight of the composition. In some embodiments, the anhydrous rapamycin gel composition disclosed herein comprises tetrahydrofurfurylalcohol polyethylene glycol ether as the one or more solvent in an amount of about 5% to about 30% of the total weight of the composition. In some embodiments, the anhydrous rapamycin gel composition disclosed herein comprises tetrahydrofurfurylalcohol polyethylene glycol ether as the one or more solvent in an amount of about 5% to about 25% of the total weight of the composition. In some embodiments, the anhydrous rapamycin gel composition disclosed herein comprises tetrahydrofurfurylalcohol polyethylene glycol ether as the one or more solvent in an amount of about 5% to about 20% of the total weight of the composition. In some embodiments, the anhydrous rapamycin gel composition disclosed herein comprises tetrahydrofurfurylalcohol polyethylene glycol ether as the one or more solvent in an amount of about 5% to about 15% of the total weight of the composition. In some embodiments, the anhydrous rapamycin gel composition disclosed herein comprises tetrahydrofurfurylalcohol polyethylene glycol ether as the one or more solvent in an amount of about 5% to about 10% of the total weight of the composition.

In some embodiments, the anhydrous rapamycin gel composition disclosed herein comprises butylene glycol as the one or more solvent in an amount of about 5% to about 55% of the total weight of the composition. In some embodiments, the anhydrous rapamycin gel composition disclosed herein comprises butylene glycol as the one or more solvent in an amount of about 5% to about 50% of the total weight of the composition. In some embodiments, the anhydrous rapamycin gel composition disclosed herein comprises butylene glycol as the one or more solvent in an amount of about 5% to about 45% of the total weight of the composition. In some embodiments, the anhydrous rapamycin gel composition disclosed herein comprises butylene glycol as the one or more solvent in an amount of about 5% to about 40% of the total weight of the composition. In some embodiments, the anhydrous rapamycin gel composition disclosed herein comprises butylene glycol as the one or more solvent in an amount of about 5% to about 35% of the total weight of the composition. In some embodiments, the anhydrous rapamycin gel composition disclosed herein comprises butylene glycol as the one or more solvent in an amount of about 5% to about 30% of the total weight of the composition. In some embodiments, the anhydrous rapamycin gel composition disclosed herein comprises butylene glycol as the one or more solvent in an amount of about 5% to about 25% of the total weight of the composition. In some embodiments, the anhydrous rapamycin gel composition disclosed herein comprises butylene glycol as the one or more solvent in an amount of about 5% to about 20% of the total weight of the composition. In some embodiments, the anhydrous rapamycin gel composition disclosed herein comprises butylene glycol as the one or more solvent in an amount of about 5% to about 15% of the total weight of the composition. In some embodiments, the anhydrous rapamycin gel composition disclosed herein comprises butylene glycol as the one or more solvent in an amount of about 5% to about 10% of the total weight of the composition.

In some embodiments, the anhydrous rapamycin gel composition disclosed herein comprises diethylene glycol as the one or more solvent in an amount of about 5% to about 55% of the total weight of the composition. In some embodiments, the anhydrous rapamycin gel composition disclosed herein comprises diethylene glycol as the one or more solvent in an amount of about 5% to about 50% of the total weight of the composition. In some embodiments, the anhydrous rapamycin gel composition disclosed herein comprises diethylene glycol as the one or more solvent in an amount of about 5% to about 45% of the total weight of the composition. In some embodiments, the anhydrous rapamycin gel composition disclosed herein comprises diethylene glycol as the one or more solvent in an amount of about 5% to about 40% of the total weight of the composition. In some embodiments, the anhydrous rapamycin gel composition disclosed herein comprises diethylene glycol as the one or more solvent in an amount of about 5% to about 35% of the total weight of the composition. In some embodiments, the anhydrous rapamycin gel composition disclosed herein comprises diethylene glycol as the one or more solvent in an amount of about 5% to about 30% of the total weight of the composition. In some embodiments, the anhydrous rapamycin gel composition disclosed herein comprises diethylene glycol as the one or more solvent in an amount of about 5% to about 25% of the total weight of the composition. In some embodiments, the anhydrous rapamycin gel composition disclosed herein comprises diethylene glycol as the one or more solvent in an amount of about 5% to about 20% of the total weight of the composition. In some embodiments, the anhydrous rapamycin gel composition disclosed herein comprises diethylene glycol as the one or more solvent in an amount of about 5% to about 15% of the total weight of the composition. In some embodiments, the anhydrous rapamycin gel composition disclosed herein comprises diethylene glycol as the one or more solvent in an amount of about 5% to about 10% of the total weight of the composition.

In some embodiments, the anhydrous rapamycin gel composition disclosed herein comprises triethylene glycol as the one or more solvent in an amount of about 5% to about 55% of the total weight of the composition. In some embodiments, the anhydrous rapamycin gel composition disclosed herein comprises triethylene glycol as the one or more solvent in an amount of about 5% to about 50% of the total weight of the composition. In some embodiments, the anhydrous rapamycin gel composition disclosed herein comprises triethylene glycol as the one or more solvent in an amount of about 5% to about 45% of the total weight of the composition. In some embodiments, the anhydrous rapamycin gel composition disclosed herein comprises triethylene glycol as the one or more solvent in an amount of about 5% to about 40% of the total weight of the composition. In some embodiments, the anhydrous rapamycin gel composition disclosed herein comprises triethylene glycol as the one or more solvent in an amount of about 5% to about 35% of the total weight of the composition. In some embodiments, the anhydrous rapamycin gel composition disclosed herein comprises triethylene glycol as the one or more solvent in an amount of about 5% to about 30% of the total weight of the composition. In some embodiments, the anhydrous rapamycin gel composition disclosed herein comprises triethylene glycol as the one or more solvent in an amount of about 5% to about 25% of the total weight of the composition. In some embodiments, the anhydrous rapamycin gel composition disclosed herein comprises triethylene glycol as the one or more solvent in an amount of about 5% to about 20% of the total weight of the composition. In some embodiments, the anhydrous rapamycin gel composition disclosed herein comprises triethylene glycol as the one or more solvent in an amount of about 5% to about 15% of the total weight of the composition. In some embodiments, the anhydrous rapamycin gel composition disclosed herein comprises triethylene glycol as the one or more solvent in an amount of about 5% to about 10% of the total weight of the composition.

Preferably, the anhydrous rapamycin gel composition disclosed herein described herein comprises one or more solvents selected from polyethylene glycol, isopropyl alcohol, diisopropyl adipate, glycerol, and combinations thereof. Preferably, if polyethylene glycol is included, polyethylene glycol is present in an amount of about 40% to about 60% of the total weight of the composition. Preferably, if isopropyl alcohol is included, isopropyl alcohol is present in an amount of about 10% to about 20% of the total weight of the composition. Preferably, if diisopropyl adipate is included, diisopropyl adipate is present in an amount of about 10% to about 20% of the total weight of the composition. Preferably, if glycerol is included, glycerol is present in an amount of about 5% to about 20% of the weight of the total composition.

### Gelling Agent

In some embodiments, the anhydrous rapamycin gel composition disclosed herein comprises a gelling agent present in an amount of about 0.1% to about 5% of the total weight of the composition. In some embodiments, the anhydrous rapamycin gel composition disclosed herein comprises a gelling agent present in an amount of about 0.1% to about 4.5% of the total weight of the composition. In some embodiments, the anhydrous rapamycin gel composition disclosed herein comprises a gelling agent present in an amount of about 0.1% to about 4% of the total weight of the composition. In some embodiments, the anhydrous rapamycin gel composition disclosed herein comprises a gelling agent present in an amount of about 0.1% to about 3.5% of the total weight of the composition. In some embodiments, the anhydrous rapamycin gel composition disclosed herein comprises a gelling agent present in an amount of about 0.1% to about 3% of the total weight of the composition. In some embodiments, the anhydrous rapamycin gel composition disclosed herein comprises a gelling agent present in an amount of about 0.1% to about 2.5% of the total weight of the composition. In some embodiments, the anhydrous rapamycin gel composition disclosed herein comprises a gelling agent present in an amount of about 0.1% to about 2% of the total weight of the composition. In some embodiments, the anhydrous rapamycin gel composition disclosed herein comprises a gelling agent present in an amount of about 0.1% to about 1.5% of the total weight of the composition. Preferably, the anhydrous rapamycin gel composition disclosed herein comprises a gelling agent present in an amount of about 0.1% to about 1% of the total weight of the composition.

In some embodiments, the anhydrous rapamycin gel composition disclosed herein comprises a gelling agent selected from hydroxypropyl cellulose, carbomer 981, carbomer 934P, glyceryl tris 12-hydroxy stearate, hydroxy stearin, propylene carbonate, polyvinyl pyrrolidine, and combinations thereof. In some embodiments, the anhydrous rapamycin gel composition disclosed herein comprises a gelling agent, such as poloxamers and carbomers. Non-limiting examples of poloxamers are poloxamer P-188, poloxamer P-138, poloxamer P-237, poloxamer P-288, poloxamer P-124, poloxamer P-338, and poloxamer P-407. Other block copolymers, such as poly(ethylene glycol/DL lactide Co-glyceride) poly(.quadrature.-caprolactum), and hydroxypropyl cellulose (KLUCEL®), glyceryl tris 12-hydroxy stearate, hydroxy stearin, propylene carbonate, polyvinyl pyrolidine can also be used as gelling agents. Non-limiting examples of carbomers that may be used are carbomer 981, carbomer 934, carbomer 934P, carbomer 940, carbomer 941, carbomer 1342, polycarbophil, and calcium polycarbophil. In a preferred embodiment, the gelling agent is selected from hydroxypropyl cellulose, carbomer 981, carbomer 934P, glyceryl tris 12-hydroxy stearate, hydroxy stearin, propylene carbonate, polyvinyl pyrolidine, and combinations thereof. Preferably, the anhydrous rapamycin gel composition disclosed herein comprises hydroxypropyl cellulose in an amount of about 0.1% to about 1% of the total weight of the composition.

### Antioxidants

In some embodiments, the anhydrous rapamycin gel composition disclosed herein comprises an antioxidant present in an amount of about 0.001% to about 1%, about 0.001% to about 0.5%, about 0.001% to about 0.1%, about 0.001% to about 0.05%, or about 0.001% to about 0.01% of the total weight of the composition. In some embodiments, the anhydrous rapamycin gel composition disclosed herein comprises an antioxidants, such as ascorbic acid, vitamin E and its derivatives, α-tocopherol, Ψ-tocopherol, Δ-tocopherol, ascorbyl palmitate, propyl gallate (PG), octyl gallate, dodecyl gallate, butylated hydroxy anisole (BHA), butylated hydroxy toluene (BHT), D-α-tocopheryl polyethylene glycol 1000 succinate, or combinations thereof. Preferably, the anhydrous rapamycin gel composition disclosed herein comprises a mixture of antioxidants comprising propyl gallate (PG), ascorbyl palmitate, and α-tocopherol. Preferably, the total amount of antioxidant in the anhydrous rapamycin gel composition disclosed herein is about 0.05% to about 0.1% of the total weight of the composition. Preferably, the anhydrous rapamycin gel composition disclosed herein comprises propyl gallate (PG) in an amount of about 0.01% to about 0.1% of the total weight of the composition. Preferably, the anhydrous rapamycin gel composition disclosed herein comprises ascorbyl palmitate about 0.01% to about 0.05% of the total weight of the composition. Preferably, the anhydrous rapamycin gel composition disclosed herein comprises α-tocopherol about 0.001% to about 0.01% of the total weight of the composition.

### Formulations

In some embodiments, the anhydrous rapamycin gel composition disclosed herein comprises rapamycin present at about 3.9% of the weight of the total composition, isopropyl alcohol present at about 15% of the weight of the total composition, polyethylene glycol 400 present at about 54.9% of the weight of the total composition, diisopropyl adipate present at about 15% of the weight of the total composition, glycerol present at about 10% of the weight of the total composition, hydroxypropyl cellulose present at about 0.75% of the weight of the total composition, propyl gallate present at about 0.05% of the weight of the total composition, ascorbyl palmitate present at about 0.02% of the weight of the total composition, alpha-tocopherol present at about 0.002% of the weight of the total composition, and a buffer. In some embodiments, the buffer is citric acid and is present in an amount of less than 0.1% of the weight of the total composition.

In some embodiments, the anhydrous rapamycin gel composition disclosed herein comprises rapamycin present at about 3.9% of the weight of the total composition, isopropyl alcohol present at about 15% of the weight of the total composition, polyethylene glycol 400 present at about 54.9% of the weight of the total composition, diisopropyl adipate present at about 15% of the weight of the total composition, glycerol present at about 10% of the weight of the total composition, hydroxypropyl cellulose present at about 0.75% of the weight of the total composition, propyl gallate present at about 0.05% of the weight of the total composition, ascorbyl palmitate present at about 0.02% of the weight of the total composition, alpha-tocopherol present at about 0.002% of the weight of the total composition, and citric acid.

Preferably, the anhydrous rapamycin gel composition disclosed herein does not contain water. In some embodiments, the anhydrous rapamycin gel composition disclosed herein contains substantially no water. In some embodiments, the anhydrous rapamycin gel compositions disclosed herein contain less than 1% of water in the total composition. In some embodiments, the anhydrous rapamycin gel compositions disclosed herein contain less than 0.5% of water in the total composition. In some embodiments, the anhydrous rapamycin gel compositions disclosed herein contain less than 0.1% of water in the total composition.

Preferably, the anhydrous rapamycin gel compositions disclosed herein does not comprise any penetration enhancers.

In some embodiments, a hand pump may be used to dispense rapamycin anhydrous compositions. For example, the hand pump may be configured to dispense the required dose of rapamycin within a tolerance specified by a corresponding label approved by a government regulatory agency. The hand pump may deliver 0.5-10 mL of the composition per pump action, such as 1, 2, 3, 4, or 5 mL of the composition per pump action. In some embodiments, the rapamycin compositions may be packaged along with a pharmaceutically acceptable hand pump.

In some embodiments, metering airless dose pumps may be used to dispense the anhydrous compositions disclosed herein. Airless type dispensing systems typically have two methods of dispensing the product, either by using a collapsible bag type design or by using a follower piston-type design. With the collapsible bag type design, a collapsing bag is attached to the dispensing pump, which progressively collapses as the contents are removed. In the piston-type design, a rigid container, usually cylindrical or oval in form, has a follower piston that progressively reduces the container volume as product is drawn out by the dispensing pump. In some embodiments, the rapamycin anhydrous compositions will be dispensed using an airless pouch, pump-actuating, container system.

In some embodiments, administration of the rapamycin anhydrous gel composition disclosed herein achieves a Cmax of about 120-990 micromolar, about 120-900 micromolar, about 120-800 micromolar, about 120-600 micromolar, about 120-400 micromolar or about 120-200 micromolar in the epidermis. In some embodiments, administration of the rapamycin anhydrous gel composition disclosed herein achieves a Cmax of about 36-350 micromolar, about 36-300 micromolar, about 36-250 micromolar, about 36-200 micromolar, or about 36-100 micromolar in the dermis. In some embodiments, the rapamycin anhydrous gel composition disclosed herein achieves a Tmax of about 15-24 hours, about 15-20 hours, or about 15-18 hours in the epidermis.

In some embodiments, the viscosity of the rapamycin anhydrous gel composition disclosed herein is generally that of a thick liquid or gel but can reach a paste like consistency. Generally, the viscosity is a minimum of about 5,000, 10,000 or 15,000 preferably about 20,000 to a maximum of about 12,000,000, 2,000,000 or even about 600,000 cP.

In some embodiments, the rapamycin in the rapamycin anhydrous gel composition disclosed herein is stable for extended periods of time. For example, in some embodiments, rapamycin in the rapamycin anhydrous gel composition disclosed herein is stable at temperature ranges from about 4° C. to about 50° C. for a period of 12-36 months. In some embodiments, rapamycin in the rapamycin anhydrous gel composition disclosed herein is stable at temperature ranges from about 4° C. to about 45° C. for a period of 12-36 months. In some embodiments, rapamycin in the rapamycin anhydrous gel composition disclosed herein is stable at temperature ranges from about 4° C. to about 40° C. for a period of 12-36 months. In some embodiments, rapamycin in the rapamycin anhydrous gel composition disclosed herein is stable at temperature ranges from about 4° C. to about 35° C. for a period of 12-36 months. In some embodiments, rapamycin in the rapamycin anhydrous gel composition disclosed herein is stable at temperature ranges from about 4° C. to about 30° C. for a period of 12-36 months.

### EXAMPLE

### Example 1: A Multicenter, Phase 2b, Randomized, Double-Blind, Stratified, Vehicle-Controlled Study Evaluating the Safety and Efficacy of Sirolimus 3.9% Topical Gel in the Prevention of BCCs in Patients with Gorlin Syndrome

Primary efficacy objective: Compare the number of new biopsy confirmed BCCs that develop on the skin over a 12-month period between the treatment arms, active and placebo. The primary efficacy endpoint is the total number of new biopsy confirmed BCCs appearing on the skin within 6 months.

Primary safety objective: Determine the incidence of dermatological, treatment emergent Adverse Events after treatment with Sirolimus 3.9% Topical Gel. The primary safety endpoint is the incidence of dermatological, treatment emergent Adverse Events after treatment with Sirolimus 3.9% Topical Gel.

Secondary efficacy objective: Compare the disease burden of biopsy confirmed BCCs over a 12 month period between treatment arms, active and placebo. Disease burden endpoints: Percentage of participants developing 2 or more new biopsy confirmed BCCs on skin. Percentage of participants developing 1 or more new biopsy confirmed BCCs on skin. Total number of new biopsy confirmed BCCs on skin within 9 months. Total number of new biopsy confirmed BCCs on skin within 6 months.

Secondary safety objective: Evaluate safety and tolerability of PTX-022 over 6 months of treatment. Safety and tolerability endpoints: Treatment emergent adverse events, including skin reactions: Changes from baseline for vital signs and adverse events related to clinical laboratory assessments: Participant compliance.

Exploratory objectives: Compare patient-reported disease symptoms and impact on quality of life over a 12-month period between the treatment arms, active and placebo. If present at baseline, compare targeted lesion (potential BCC) size to size up to Month 12. Compare the number of new clinically suspicious BCCs that develop on the skin over a 12-month period between the treatment arms, active and placebo. Exploratory endpoints: Change in Advanced Basal Cell Carcinoma Index (aBCCdex) and DLQI between Baseline and Month 6: If present at Baseline, change in the sum of the longest diameter of targeted lesions, between Baseline and Month 12: Time to first new biopsy confirmed BCC: Time to second new biopsy confirmed.

Overall design: A multicenter, Phase 2b, randomized, double-blind, stratified, vehicle-controlled study.

This is a multicenter, double-blind, randomized, vehicle-controlled study to assess the safety and efficacy of PTX-022 in patients with Gorlin syndrome. To be eligible for the study, trial participants must meet study entry criteria, including diagnostic criteria for GS, presence of clinically typical BCCs on the skin and body and absence of concurrent treatment with other investigational or specified treatments.

Approximately 60 trial participants who meet study entry criteria will be randomized in a 1:1 ratio to receive PTX-022 or vehicle. Study drug will be applied topically to the entire skin once daily for 48 weeks of treatment. All trial participants will be instructed to avoid exposure to direct sunlight and to continue their use of sunscreens to minimize their exposure to ultraviolet radiation.

Assessments that support the trial objectives will be performed at clinic visits. The investigator will perform a clinical evaluation of the skin at all clinic visits, as well as will take full face photographs to aid in tracking. Any clinically suspicious BCC will be biopsied. Any new biopsy-confirmed BCC that is confirmed between the Month 2 and Month 12 treatment period will be counted in support of the primary endpoint. At the end of the treatment period, all trial participants will be contacted 4 weeks after their last treatment to collect any trial-related information, especially adverse events.

Participants will have at least 8 visits (6 in clinic, 2 via telephone) which include Screening (up to 9 months permitted for wash out of exclusionary medications), Baseline (Day 0), Telephone Visit (Week 2+2 days) Month 1 (Week 4+2 weeks), Month 2 (Week 8+2 weeks), Month 4 (Week 16+2 weeks), Month 6 (Week 24+2 weeks), and Telephone visit Follow-Up (Week 28+1 week).

Each subject will be in the treatment period approximately 32 weeks from the time the subject signs ICF through the last protocol visit.

INTRODUCTION: Sirolimus 3.9% topical gel is being developed for the prevention of Basal Cell Carcinomas in patients with Gorlin syndrome (GS) in subjects aged ≥18 and older. GS is also referred to by several other names, including Gorlin-Goltz Syndrome, Basal Cell Nevus Syndrome (BCNS), and Nevoid Basal Cell Carcinoma Syndrome (NBCCS).+

GS is a rare autosomal-dominant genetic disease primarily caused by aberrant Hedgehog signaling through germline mutations in the PTCH1 gene. Patients with this disorder are predisposed to develop basal cell carcinomas, or BCCs, malignant cancers of the outer layer of the skin most commonly appearing on the face, chest, neck and back, at a significantly greater rate than people of similar skin type who do not have the disease. Recent research has indicated that patients report a mean of 257 BCCs over their lifetime (Solis 2017). Accordingly, this condition requires continual lifetime management to avoid progression to advanced or fatal metastatic BCCs. GS BCCs are typically treated through surgical removals which can be disfiguring and significantly impact patient quality of life.

Based on the need to prevent the burdensome development of BCCs, we designed this phase 2b study to evaluate PTX-022 in the prevention of new, biopsy confirmed BCCs on the face. The primary efficacy objective of this study is to compare the number of new biopsy confirmed BCCs (bcBCCs) that develop over a 12-month period between the treatment arms, active and placebo. bcBCCs are those that have a confirmed histopathological diagnosis of BCC. Secondary efficacy objectives will include disease burden assessments at various timepoints.

The primary safety objective is to determine the incidence of dermatological, treatment emergent adverse events with secondary safety objectives to include the assessment of all treatment emergent adverse events, patient compliance and changes from baseline physical exams, vital signs and clinical laboratory assessments.

GS patients can present with hundreds of BCCs over their lifetime: consequently, GS patients often experience a significantly diminished quality of life. This program will focus on the prevention of the cutaneous manifestations of GS, the BCCs, which are described by patients as the most debilitating feature of their disease.

PTCH1 Mutation Causes Aberrant Hedgehog Signaling, Driving BCC Growth: Growth of BCCs in GS patients is primarily driven by aberrant activation of the hedgehog signaling pathway. Hedgehog signaling can proceed through two distinct pathways: canonical and non-canonical. Non-canonical hedgehog signaling proceeds via the mTOR pathway through GLI1 in response to stimuli, such as TNF-a. During canonical pathway signaling hedgehog binding to PTCH1 induces movement of SMO to the ciliary membrane leading to inhibition of SUFU, activation of GL1 and subsequent transcription of hedgehog-related genes.

Loss of function mutations in the tumor suppressor gene PTCH1 (9q22.1q31), which encodes the receptor of the sonic hedgehog ligand, are the root cause of GS. Approximately 70% of GS patients have mutations in the PTCH1 gene. PTCH1 regulates growth and development of normal tissue by repressing the transcription of genes encoding proteins belonging to transforming growth factor beta (TGF-β). Patients without PTCH1 mutations may have a loss of function mutation in the SUFU gene, which also regulates the hedgehog signaling pathway.

Diagnosis and available treatments: BCCs are the predominant cutaneous manifestation of GS and one of the few features of GS that is reversible. BCCs can be macroscopic or microscopic, and most frequently appear on the face. In GS patients, BCCs usually start developing in adolescence, with a median age of onset of 20 years. At initial presentation, GS patients have a median of 8 BCCs, and can have as many as hundreds. There are eight basal cell carcinoma subtypes: Superficial, Nodular (solid), Micronodular, Infiltrative, Fibroepithelial (Pinkus tumor), Basal cell carcinoma with adnexal differentiation, Basosquamous carcinoma, and Keratotic.

Basal cell carcinomas may be plaque-like or nodular with a waxy, translucent appearance, often with ulceration and telangiectasia. Most BCCs are identified via clinical examination, dermatoscopy, optical coherence tomography and/or confocal laser scanning microscopy of the skin. These modalities are also useful presurgical assessments and to evaluate response after treatment. Diagnosis is confirmed via histopathology. Clinically suspicious lesions are commonly biopsied using shave or punch biopsies to collect the pathology samples.

GS patients develop BCCs in greater frequency in areas of the skin that are exposed to the sun, such as face, scalp, trunk and arms, or other radiation. Therefore, patients with GS need to limit their exposure to radiation given this known relationship between radiation exposure and the development of BCCs in the radiation field.

The current standard of care for GS patients consists only of surgical removal of BCCs. To avoid progression of BCCs to advanced or fatal metastatic BCC, GS patients usually undergo multiple surgeries to remove BCCs.

Another treatment option is the use of oral Hh inhibitors, like Vismodegib. Efforts to inhibit the Hh pathway clinically have only been partially successful. BCC cells treated with SMO inhibitors (SMOi) can rapidly bypass inhibition or adopt an alternative identity that does not rely on the original oncogenic driver for survival. Vismodegib-tolerant residual tumor cells persist in the skin of GS patients, leading to relapse after discontinuation of treatment. In addition, oral Hh inhibitors are associated with a highly unacceptable side-effect profile. Collectively, these results indicate that Hh signaling blockade is not sufficient for permanent eradication of BCCs and that research strategies directed at finding additional molecular targets are needed to achieve sustained remission and cure of these tumors.

Unmet Need in the Gorlin Syndrome Patient Population: Physicians report that GS patients experience bleeding, oozing, pain, and odors from untreated lesions. In addition to the incapacitating nature of the disease, it has significant impact on the emotional well-being of patients. GS patients continue to live with a burden of multiple lesions, ulcerations potentially leading to infection, disfigurement, and impaired function, collectively leading to emotional suffering. A comprehensive study of 32 GS patients using the Skindex-29 survey to measure skin-related quality of life found that GS patients are severely impaired emotionally. Therefore, GS is a debilitating disease in need of new management approaches.

A topically formulated mTOR inhibitor could potentially help to substantially reduce the morbidity associated with the multiple surgical procedures needed to remove BCCs in GS patients. Simultaneously this would obviate the risk of the highly unacceptable side-effect profile of orally administered Hh inhibitors that has caused half or more of GS patients to discontinue these drugs despite their remarkable anti-tumor efficacy.

INCLUSION CRITERIA: Participants are eligible to be included in the study only if all the following criteria apply: At the Screening Visit: 1. The participant must be age at least 18 years of age at the Screening Visit. 2. The participant must provide written informed consent/assent prior to any study procedures. 3. The participant must meet diagnostic criteria for GS including major criterion #3a plus 1 additional major criterion or plus 2 additional minor criteria listed below. Major criteria: a. >2 histologically confirmed BCCs or 1 for participant under age 20, b. Odontogenic keratocysts of the jaw confirmed histologically, c. >3 palmar and/or plantar pits seen at the Screening Visit, d. Bilamellar calcification of the falx cerebri present at less than 20 years of age, e. Fused, bifid, or markedly splayed ribs, f. First degree relative with GS, g. Patched-1 gene(PTCH1) mutation predicted to be of functional significance in normal tissue. Minor criteria: h. Macrocephaly, i. Congenital malformations including frontal bossing, cleft lip or palate, “coarse face”, moderate to severe hypertelorism. j. Skeletal abnormalities detectable clinically: Sprengel deformity, marked pectus deformity, or marked finger syndactyly, k. Skeletal abnormalities detectable radiographically: bridging of the sella turcica: vertebral abnormalities such as hemivertebrae, fusion or elongation of the vertebral bodies: modeling defects of the hands and feet: flame shaped lucencies of the hands or feet. 1. Ovarian fibroma, m. Medulloblastoma. 4. The participant is willing to have blood collected for safety and PK testing. 5. The participant is willing to abstain from application of a non-study topical medication (prescription or over the counter) to face for the duration of the trial. Moisturizers and emollients are allowed. Participant will be encouraged to use their preferred sunscreen with a sun protector factor (SPF) of at least 30 daily on all exposed skin sites. 6. Participants of childbearing potential must have a negative urine pregnancy test to participate in the study, a. Participants of childbearing potential must agree to use a medically acceptable, highly effective form of birth control such as birth control pills, depo-progesterone injections, a vaginal hormonal contraceptive ring, a barrier contraceptive such as a condom with spermicide cream or gel, diaphragms or cervical cap with spermicide cream or gel, or an intrauterine device (IUD) for the entire duration of the study and for an additional 4-week period after their last dose of study medication. 7. The participant is willing to forego treatment of BCCs with anything other than the study IP except when the Investigator believes that delay of treatment of a BCC potentially might compromise the health of the subject. During the trial, the only allowed form of BCC treatment is surgical. At Visit 1: 8. The participant can have no more than three biopsy confirmed BCCs on the face, excluding periorbital skin, at the Baseline Visit/Visit 1. Participants are not required to have BCCs visible on the face at Visit 1 to enter the trial. 9. The participant must have 10 BCCs present on the face, scalp and/or neck (clinically diagnosed and/or biopsy confirmed) within 24 months prior to Randomization.

### Efficacy Assessments

Biopsy: In accordance with standard of care treatment guidelines (NCCN), biopsies will be performed on all clinically suspicious lesions by qualified personnel. Biopsies must be obtained after photographs of the face have been taken. Following standard clinical practice at the site, biopsy samples will be read by local pathology laboratory.

BCC Tracking via Digital Photography: The investigator will perform a clinical evaluation to identify all BCCs on the face and, in accordance with the photography manual, lesions will be tracked by digital photography. These photos will be obtained at every clinic visit.

1. A method of treating Gorlin Syndrome in a subject in need thereof comprising topically administering to the affected areas of the subject once daily a therapeutically effective amount of an anhydrous rapamycin gel composition, wherein treatment prevents or decreases the number new basal cell carcinomas (BCCs) that develop on the skin of the subject.

2. The method of claim 1, wherein following administration of the anhydrous rapamycin gel composition the subject does not develop a new BCC on the skin in a 6 month period once treatment has started: the subject develops no more than 2 new BCCs on the skin in about a 6 month period once treatment has started; the size of a BCC on the skin lesion has decreased: the sum diameter of a BCC lesion on the skin has decreased; or a combination thereof.

3. The method of claim 1, wherein following administration of the anhydrous rapamycin gel composition the subject does not develop a new BCC on the skin in a 12 month period once treatment has started: the subject develops no more than 2 new BCCs on the skin in about a 12 month period once treatment has started; the subject develops no more than 1 new BCC on the skin in about a 12 month period once treatment has started; or a combination thereof.

4. (canceled)

5. The method of claim 1, wherein the anhydrous rapamycin gel composition comprises about 0.1% to about 5% of rapamycin, about 80% to about 99% of one or more solvents, about 0.1% to about 5% of a gelling agent, and about 0.001% to about 1% of an antioxidant.

6. The method of claim 5, wherein the anhydrous rapamycin gel composition comprises:
about 3.9% of rapamycin,
about 15% isopropyl alcohol,
about 54.9% polyethylene glycol 400,
about 15% diisopropyl adipate,
about 10% glycerol,
about 0.75% hydroxypropyl cellulose,
about 0.05% propyl gallate,
about 0.02% ascorbyl palmitate,
about 0.002% alpha-tocopherol, and
citric acid.

7. The method of claim 1, wherein the anhydrous rapamycin gel composition is administered once daily for at least 24 weeks.

8. (canceled)

9. (canceled)

10. The method of claim 1, wherein the BCC is selected from the group consisting of: Superficial, Nodular (solid), Micronodular, Infiltrative, Fibroepithelial (Pinkus tumor), Basal cell carcinoma with adnexal differentiation, Basosquamous carcinoma, Keratotic, or combinations thereof

11. (canceled)

12. (canceled)

13. A method of decreasing the risk of progression to basal cell carcinoma in a subject with Gorlin Syndrome comprising topically administering to the skin of the subject once daily a therapeutically effective amount of an anhydrous rapamycin gel composition, wherein treatment prevents or decreases the number new basal cell carcinomas (BCCs) that develop on the skin of the subject.

14. The method of claim 13, wherein following administration of the anhydrous rapamycin gel composition the subject does not develop a new BCC on the skin in a 6 month period once treatment has started: the subject develops no more than 2 new BCCs on the skin in about a 6 month period once treatment has started: the size of a BCC lesion on the skin has decreased; the sum diameter of a BCC lesion on the skin has decreased; or a combination thereof.

15. The method of claim 13, wherein following administration of the anhydrous rapamycin gel composition the subject does not develop a new BCC on the skin in a 12 month period once treatment has started; the subject develops no more than 2 new BCCs on the skin in about a 12 month period once treatment has started; the subject develops no more than 1 new BCC on the skin in about a 12 month period once treatment has started; or a combination thereof.

16. (canceled)

17. The method of claim 13, wherein the anhydrous rapamycin gel composition comprises about 0.1% to about 5% of rapamycin, about 80% to about 99% of one or more solvents, about 0.1% to about 5% of a gelling agent, and about 0.001% to about 1% of an antioxidant.

18. The method of claim 17, wherein the anhydrous rapamycin gel composition comprises:
about 3.9% of rapamycin,
about 15% isopropyl alcohol,
about 54.9% polyethylene glycol 400,
about 15% diisopropyl adipate,
about 10% glycerol,
about 0.75% hydroxypropyl cellulose,
about 0.05% propyl gallate,
about 0.02% ascorbyl palmitate,
about 0.002% alpha-tocopherol, and
citric acid.

19. The method of claim 13, wherein the anhydrous rapamycin gel composition is administered once daily for at least 24 weeks.

20. (canceled)

21. (canceled)

22. The method of claim 13, wherein the BCC is selected from the group consisting of: Superficial, Nodular (solid), Micronodular, Infiltrative, Fibroepithelial (Pinkus tumor), Basal cell carcinoma with adnexal differentiation, Basosquamous carcinoma, Keratotic, or combinations thereof

23. (canceled)

24. (canceled)

25. A method of decreasing the risk of developing basal cell carcinomas in a subject with Gorlin Syndrome comprising topically administering to the skin of the subject once daily a therapeutically effective amount of an anhydrous rapamycin gel composition, wherein treatment prevents or decreases the number new basal cell carcinomas (BCCs) that develop on the skin of the subject.

26. The method of claim 25, wherein following administration of the anhydrous rapamycin gel composition the subject does not develop a new BCC on the skin in a 6 month period once treatment has started: the subject develops no more than 2 new BCCs on the skin in about a 6 month period once treatment has started: the size of a BCC lesion on the skin has decreased; the sum diameter of a BCC lesion on the skin has decreased; or a combination thereof.

27. The method of claim 25, wherein following administration of the anhydrous rapamycin gel composition the subject does not develop a new BCC on the skin in a 12 month period once treatment has started: the subject develops no more than 2 new BCCs on the skin in about a 12 month period once treatment has started: the subject develops no more than 1 new BCC on the skin in about a 12 month period once treatment has started; or a combination thereof.

28. (canceled)

29. The method of claim 25, wherein the anhydrous rapamycin gel composition comprises about 0.1% to about 5% of rapamycin, about 80% to about 99% of one or more solvents, about 0.1% to about 5% of a gelling agent, and about 0.001% to about 1% of an antioxidant.

30. The method of claim 25, wherein the anhydrous rapamycin gel composition comprises:
about 3.9% of rapamycin,
about 15% isopropyl alcohol,
about 54.9% polyethylene glycol 400,
about 15% diisopropyl adipate,
about 10% glycerol,
about 0.75% hydroxypropyl cellulose,
about 0.05% propyl gallate,
about 0.02% ascorbyl palmitate,
about 0.002% alpha-tocopherol, and
citric acid.

31. The method of claim 25, wherein the anhydrous rapamycin gel composition is administered once daily for at least 24 weeks.

32. (canceled)

33. (canceled)

34. The method of claim 25, wherein the BCC is selected from the group consisting of Superficial, Nodular (solid), Micronodular, Infiltrative, Fibroepithelial (Pinkus tumor), Basal cell carcinoma with adnexal differentiation, Basosquamous carcinoma, Keratotic, or combinations thereof.

35. (canceled)

36. (canceled)
